(19) United States
(12) Patent Application Publication
(10) Pub. No.: US 2003/0144171 A1
(43) Pub. Date: Jul. 31, 2003

(54) Flowable mixtures of isethionate and alcohol

(75) Inventors: Torsten Henning, Bad Soden (DE); Waltraud Simsch, Kelkheim (DE)

Correspondence Address: Clariant Corporation, Industrial Property Department, 4000 Monroe Road, Charlotte, NC, 28205, US

(73) Assignee: Clariant GmbH

(21) Appl. No.: 10/352,811

(22) Filed: Jan. 28, 2003

(30) Foreign Application Priority Data
Jan. 31, 2002 (DE) 10203924.0

(51) Int. Cl. A61K 7/075

(52) U.S. Cl. 510/426; 510/428; 510/505; 510/119

(57) ABSTRACT

The invention relates to flowable mixtures comprising isethionate in combination with polyethylene glycols and to their use in liquid products without a further heating or solubilization operation.

The invention relates to mixtures comprising isethionates which are flowable at room temperature. Isethionates are known as mild surfactants used widely in cosmetic compositions and laundry detergents.

To date, isethionates have been available on the market exclusively as solid products which have to be dissolved hot using surfactants or solvents with high energy and time expenditure. Often, processing produces troublesome dusting of the raw material, which further increases the expenditure for use, for example suitable protective clothing is necessary or it is necessary to work in specially sealed plants.

Surprisingly, it has now been found that mixtures of isethionates and alcohols are flowable and pumpable at room temperature.

The invention provides flowable mixtures of at least one isethionate of the formula

RCOOCH₂CH₂SO₃M

where R is C₆-C₃₀-alkyl, preferably C₈-C₂₂-alkyl or C₆-C₃₀-alkenyl, preferably C₈-C₂₂-alkenyl, and M is an alkali metal, alkaline earth metal or ammonium ion, and at least one mono- or polyhydric alcohol.

Suitable alcohols are, in particular, propylene glycol and glycerol. Particular preference is given to polyethylene glycols of the formula H(OCH₂CH₂)ₙOH, where n is a number from 2 to 16.

The quantitative ratio of isethionate to alcohol can vary between 1 to 90% by weight, preferably 10 to 80% by weight: 10 to 99% by weight, preferably 20 to 90% by weight. In addition, these mixtures can also comprise one or more surfactants, preferably fatty alcohol oxethylates, or water in amounts of up to 70% by weight in each case.

A particular advantage of the mixtures according to the invention is that they comprise no or at least little water and are thus self-preserving. In this way, customers are able to use their own preserving system for the final products and are not tied to the co-supplied preservative.

In addition, the polyethylene glycol synergistically aids the mild cleansing action of the isethionate by improving the feel on the skin.

The mixtures according to the invention can be stably incorporated into liquid end-products, such as, for example, hair shampoos, shower preparations or liquid detergents without a further heating or solubilization operation.

The examples below serve to illustrate the subject-matter of the invention in more detail without limiting it thereto.

### EXAMPLES

### Example 1

40% by weight of Hostapon SCI-65 were mixed with 60% by weight of polyglycol 200 USP. The mixture remains flowable.

### Example 2

35% by weight of Hostapon SCI-85 were mixed with 65% by weight of polyglycol 300. The mixture remains flowable.

### Example 3

30% by weight of Hostapon SCI-85 were mixed with 20% by weight of Hostapon KCG and 50% by weight of polyglycol 200 USP. The mixture remains flowable.

### Example 4

1.9% by weight of Hostapon SCI-85 were mixed with 4.4% by weight of polyglycol 200 USP, 78.1% by weight of Genapol LRO liquid and 15.6% by weight of Genagen CAB at elevated temperature and cooled. The mixture remains flowable and is clear.

### Example 5

45% by weight of Hostapon SCI-85 were mixed with 27.5% by weight of polyglycol 400 and 27.5% by weight of Genapol L-3 at room temperature. The mixture remains flowable.

List of products used:

| | | |
|---|---|---|
| Polyglycol 200 USP | (Clariant GmbH) | PEG-4, polyethylene glycol |
| | | with an average molar mass of |
| | | 200 g/mol |
| Polyglycol 300 | (Clariant GmbH) | PEG-6, polyethylene glycol |
| | | with an average molar mass of |
| | | 300 g/mol |
| Polyglycol 400 | (Clariant GmbH) | PEG-8, polyethylene glycol |
| | | with an average molar mass of |
| | | 400 g/mol |
| Hostapon ® SCI-65 | (Clariant GmbH) | sodium cocoyl isethionate |
| Hostapon ® SCI-85 | (Clariant GmbH) | sodium cocoyl isethionate |
| Hostapon ® KCG | (Clariant GmbH) | sodium cocoyl glutamate |
| Genapol ® LRO | (Clariant GmbH) | alkyldiglycol ether sulfate |
| | | Na salt |
| Genapol ® L-3 | (Clariant GmbH) | laureth-3 |
| Genagen ® CAB | (Clariant GmbH) | cocoamidopropylbetaine |

We claim:

1. A flowable mixture of at least one isethionate of the formula
RCOOCH₂CH₂SO₃M
where R is C₆-C₃₀-alkyl and M is an alkali metal, alkaline earth metal or ammonium ion, and at least one mono- or polyhydric alcohol.

2. The flowable mixture as claimed in claim 1, wherein the mono- or polyhydric alcohol comprises a polyethylene glycol of the formula
H(COCH₂CH₂)ₙOH
in which n is a number from 2 to 16.

3. The flowable mixture as claimed in claim 1, comprising 1 to 99% by weight of isethionate and 10 to 99% by weight of the mono- or polyhydric alcohol.

4. The flowable mixture as claimed in claim 1, comprising 20 to 90% by weight of isethionate and 10 to 80% by weight of a mono- or polyhydric alcohol.

5. The flowable mixture as claimed in claim 1, additionally comprising up to 70% by weight of a surfactant.

6. The flowable mixture as claimed in claim 1, additionally comprising up to 70% by weight of water.

7. A method for shampooing hair comprising contacting hair with a hair shampoo comprising the flowable mixture as claimed in claim 1.

8. A hair shampoo comprising the flowable mixture of claim 1.

9. A shower preparation comprising the flowable mixture of claim 1.

10. A liquid detergent comprising the flowable mixture of claim 1.

11. The flowable mixture of claim 1 wherein R is C₈-C₂₂-alkyl or C₆-C₃₀ alkenyl.

12. The flowable mixture of claim 1 wherein R is C₈-C₂₂-alkenyl.
